(19) United States
(12) Patent Application Publication
(10) Pub. No.: US 2023/0165869 A1
(43) Pub. Date: Jun. 1, 2023

(54) DIAZINE AND TRIAZINE COMPOUNDS TO TREAT CYTOKINE STORM SYNDROME

(71) Applicant: UNIVERSITY OF GREENWICH, London (GB)

(72) Inventors: Mike LEACH, London (GB); Paul WILLIAMS, London (GB)

(73) Assignee: UNIVERSITY OF GREENWICH, London (GB)

(21) Appl. No.: 17/921,030

(22) PCT Filed: Apr. 26, 2021
(86) PCT No.: PCT/GB2021/051005

§ 371 Date: Oct. 24, 2022

(30) Foreign Application Priority Data
Apr. 24, 2020 (GB) 2006080.2

(51) Int. Cl. A61K 31/53; A61K 31/505; A61P 29/00

(52) U.S. Cl. CPC A61K 31/53; A61K 31/505; A61P 29/00

(57) ABSTRACT

The present invention relates to diazine and triazine compounds having activity as Interferon and Interleukin inhibitors, particularly Interferon-gamma, Tumour necrosis factor (TNF)-α and Interleukin-1β, 2, 4, 6, 8, 13 and 17 inhibitors, and to the compounds for use in the treatment of cytokine storm syndrome or cytokine release syndrome.

### TECHNICAL FIELD

The present invention relates to diazine and triazine compounds having activity as Interferon and Interleukin inhibitors, particularly Interferon-gamma, Tumour necrosis factor (TNF)-α and Interleukin-1β, 2, 4, 6, 8, 13 and 17 inhibitors, and to the compounds for use in the treatment of cytokine storm syndrome or cytokine release syndrome.

### BACKGROUND

WO2009090431A discloses triazines of the formula below, in which the A ring may be a sulphur containing heterocycle such as thienyl and benzothienyl, optionally substituted.

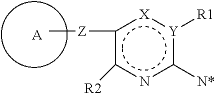

WO2009090431A further discloses triazines of the formula:

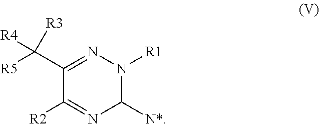

The compounds of WO2009090431A are stated to have activity as voltage dependent sodium channel blockers.

Cytokine storm syndrome (CSS) was defined by Canna and Behrens in 2012 as ‘a group of disorders representing a variety of inflammatory etiologies with the final common result of overwhelming systemic inflammation, hemodynamic instability, multiple organ dysfunction, and potentially death.’ See Pediatr Clin North Am. 2012 April; 59(2): 329-344. Cytokine storm syndrome may be triggered by a variety of factors, including infections, such as viruses, or sepsis infection (Chaudhry et al., In Vivo. 2013; 27(6): 669-684); or by treatment with certain medications (Nebelsiek et al., Recent Pat Cardiovasc Drug Discov 2012; 7: 170-4). Cytokine release syndrome refers to an adverse systemic inflammatory response to treatment with monoclonal antibodies (Winkler et al., Blood 1999; 94:2217-24). Tisoncik et al., discuss the immunopathogenesis caused by cytokine storm syndrome triggered by SARS-CoV, influenza virus, and dengue virus infections (Microbiology and Molecular Biology Reviews, March 2012 Vol. 76 No. 1, p. 16-32). Tisoncik et al., further discuss the pathology of the cytokine storm, stating that Interferon-gamma, and Interleukin (IL)-1 beta, 6, 8 and 17 are all associated with the cytokine storm, with IL-6 and IL-17 being identified as key cytokine storm mediators in gene knockout mice studies. Russell et al. review trials of interferon inhibitors and Interleukin (IL)-1, 2 and 6 inhibitors (ecancer 2020, 14:1022). They report that the increased expression of IL-2R and IL-6 in serum is expected to predict the severity of the 2019-nCoV pneumonia and the prognosis of patients and that it has been observed that COVID-19 induces high levels of IL-6 for at least 2 weeks after disease onset. The authors further conclude that IL-1 is elevated in patients infected with a coronavirus.

Rider et al. and Lin et al. discuss the compensatory mechanism observed when one specific cytokine activity or mechanism is blocked. Rider et al. (International Journal of Cell Biology, Vol. 2016, Article ID 9259646, 11 pages) state ‘The drawbacks of cytokine therapy come due to the basic properties of cytokines: (I) cytokines are pleiotropic, meaning that they affect several processes in parallel; (ii) cytokines are also known to have redundancy, meaning that the effect achieved by blocking one specific cytokine activity can be compensated by others . . . ’, while Lin et al. (Acta Biomaterialia 10 (2014) 3747-3755), discussing studies of the alleviation of wear-particle-induced osteolysis, state ‘Although blocking individual cytokines showed promising effects . . . , a clinical study in humans indicated that blocking TNF-α by neutralizing antibody did not mitigate osteolysis. This could be explained by the compensatory up-regulation of other pro-Inflammatory factors’ (emphasis added).

### SUMMARY OF THE INVENTION

The invention provides a compound of the formula (I), or a salt, tautomer or solvate thereof;

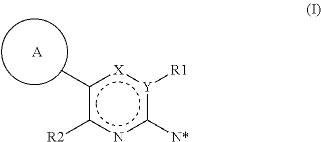

in which:

### X is N and Y is C; or

### X is C and Y is N; or

X and Y are both N:
A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆ alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆ alkylthio groups; or
A is a group

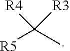

(wherein ⋅ indicates the point of attachment)
R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;
R2 is amino, C₁₋₁₀ alkyl or phenyl;
R3 is phenyl, xanthyl or naphthyl, each optionally substituted with 1 to 5 substituents selected from halogen or C₁-C₆alkoxy groups;
R4 is selected from hydrogen, C₁-C₆alkyl, C₃-C₈cycloalkyl, phenyl, xanthyl or naphthyl, wherein the phenyl or naphthyl may be optionally substituted with 2 to 5 substituents selected from halogen or C₁-C₆alkoxy groups;
R5 is hydrogen;
and
N* is ═NH when R1 is hydrogen or a substituent group; or
N* is a group NRaRb where Ra and Rb are independently H or an alkyl group; or
N* is a piperazinyl ring, optionally substituted with one or more halogen or C₁-C₆alkoxy groups; for use in the treatment of cytokine storm syndrome or cytokine release syndrome.

The Invention further provides a method of treating cytokine storm syndrome or cytokine release syndrome, comprising the step of administering to a subject in need thereof a therapeutically effective amount of a compound of formula (I) or a salt, tautomer or solvate thereof, as defined hereinabove.

The invention further provides the use of a compound of formula (I) or a salt, tautomer or solvate thereof, as defined hereinabove, in the manufacture of a medicament for use in the treatment of cytokine storm syndrome or cytokine release syndrome.

The invention further provides a pharmaceutical composition comprising a compound of formula (I) or a salt, tautomer or solvate thereof, as defined hereinabove, and one or more pharmaceutically acceptable excipients for use in the treatment of cytokine storm syndrome or cytokine release syndrome.

### BRIEF DESCRIPTION OF THE DRAWINGS

FIG. 1 illustrates the effects of compound 1 on cytokine production from stimulated human peripheral blood mononuclear cells.

FIG. 2 illustrates the inhibitory effect of compound 1 on Interleukin (IL)-1 beta.

FIG. 3 illustrates the inhibitory effect of compound 1 on interleukin (IL)-6.

FIG. 4 illustrates the inhibitory effect of compound 1 on interleukin (IL)-8.

FIG. 5 illustrates the inhibitory effect of compounds 1 on Tumour Necrosis Factor (TNF)-α.

FIG. 6 illustrates the inhibitory effect of compound 1 on interferon-gamma (IFN-γ).

FIG. 7 illustrates the inhibitory effect of compound 1 on interleukin (IL)-17A.

FIG. 8 illustrates the inhibitory effect of compound 2 on interleukin (IL)-17A.

FIG. 9 illustrates the inhibitory effect of compound 2 on Tumour Necrosis Factor (TNF)-α.

FIG. 10 illustrates the inhibitory effect of compound 2 on interferon-gamma (IFN-γ).

FIG. 11 illustrates the inhibitory effect of compound 2 on interleukin (IL)-8.

FIG. 12 illustrates the inhibitory effect of compound 2 on interleukin (IL)-6.

### DETAILED DESCRIPTION OF THE INVENTION

The compounds of the Formula (I) have been found to exhibit activity as inhibitors of interferon-gamma, Tumour necrosis factor (TNF)-α and of interleukin-1 beta, 2, 4, 6, 8, 13 or 17, and are therefore useful in the treatment of cytokine storm syndrome and cytokine release syndrome. It is believed that the activity via multiple mechanism exhibited by the compounds of Formula (I) addresses the compensatory up-regulation of other pro-inflammatory factors observed when one factor is inhibited.

As Embodiment 1 therefore, the invention provides a compound of Formula (I), or a salt, tautomer or solvate thereof,

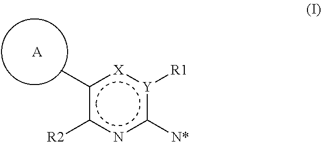

in which:

### X is N and Y is C; or

### X is C and Y is N; or

X and Y are both N:
A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆ alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆ alkylthio groups; or
A is a group

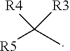

(wherein ⋅ indicates the point of attachment)
R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀ cycloalkyl, any of which is optionally substituted by hydroxy, halogen, carboxamide, halo C₁₋₆ alkyl, C₁₋₆ alkyl or C₁₋₆ alkoxy; or the Y is N and is unsubstituted;
R2 is amino, C₁₋₁₀ alkyl or phenyl;
R3 is phenyl, xanthyl or naphthyl, each optionally substituted with 1 to 5 substituents selected from halogen or C₁-C₆alkoxy groups;
R4 is selected from hydrogen, C₁-C₆alkyl, C₃-C₈cycloalkyl, phenyl, xanthyl or naphthyl, wherein the phenyl or naphthyl may be optionally substituted with 2 to 5 substituents selected from halogen or C₁-C₆alkoxy groups;
R5 is hydrogen;
and
N* is ═NH when R1 is hydrogen or a substituent group; or
N* is a group NRaRb where Ra and Rb are independently H or an alkyl group; or
N* is a piperazinyl ring, optionally substituted with one or more halogen or C₁-C₆alkoxy groups; for use in the treatment of cytokine storm syndrome or cytokine release syndrome.

As Embodiment 2, the invention provides a compound of Formula (I), or a salt, tautomer or solvate thereof, as defined in Embodiment 1, for use in the treatment of cytokine storm syndrome triggered by an infection.

As Embodiment 3, the invention provides a compound of Formula (I), or a salt, tautomer or solvate thereof, as defined in Embodiment 1, for use in the treatment of cytokine storm syndrome triggered by an infection selected from sepsis, influenza virus, a coronavirus, and dengue virus.

As Embodiment 4, the invention provides a compound of Formula (I), or a salt, tautomer or solvate thereof, as defined in Embodiment 1, for use in the treatment of cytokine storm syndrome triggered by a coronavirus.

As Embodiment 5, the invention provides a compound of Formula (I), or a salt, tautomer or solvate thereof, for use as defined in any preceding Embodiment, wherein X and Y are both N.

As Embodiment 6, the invention provides a compound of Formula (I), or a salt, tautomer or solvate thereof, for use as defined in any preceding Embodiment, wherein R1 is hydrogen.

As Embodiment 7, the invention provides a compound of Formula (I), or a salt, tautomer or solvate thereof, for use as defined in any preceding Embodiment, wherein R2 is amino.

As Embodiment 8, the invention provides a compound of Formula (I), or a salt, tautomer or solvate thereof, for use as defined in any preceding Embodiment, wherein A is a substituted 3 to 10 membered heterocycle comprising one, two or three sulphur atoms; said heterocycle having two or more substituents selected from (i) halogen; (ii) C₁₋₆ alkyl, C₂₋₆alkenyl, C₂₋₆alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆alkylthio groups.

As Embodiment 9, the invention provides a compound of formula (I), or a salt, tautomer or solvate thereof, for use as defined in any preceding Embodiment, wherein A is thienyl, or benzothienyl.

As Embodiment 10, the invention provides a compound of formula (I), or a salt, tautomer or solvate thereof, for use as defined in Embodiment 8 or 9, wherein A is substituted with one or more substituents selected from halogen, C₁₋₆alkyl, C₁₋₆alkoxy, haloC₁₄ alkyl and haloC₁₋₆alkoxy.

As Embodiment 11, the invention provides a compound of formula (I), or a salt, tautomer or solvate thereof, for use as defined in any one of Embodiments 8 to 10, wherein A is substituted with 1, 2, or 3 chlorine or bromine atoms.

As Embodiment 12, the invention provides a compound of formula (I), or a salt, tautomer or solvate thereof, for use as defined in any one of Embodiments 8 to 11, wherein the compound is selected from the group consisting of:
3,5-Diamino-6-(2-thienyl)-1,2,4-triazine;
3,5-Diamino-6-(3-thienyl)-1,2,4-triazine;
5(3)-Amino-6-(2-thienyl)-2,3(2,5)-dihydro-3(5)-imino-2-methyl-1,2,4-triazine methanesulfonate;
5(3)-Amino-6-(2-thienyl)-2,3(2,5)-dihydro-3(5)-imino-2-ethyl-1,2,4-triazine methanesulfonate;
3,5-Diamino-6-(2-thienyl)-1,2,4-triazine;
3,5-Diamino-6-(3-thienyl)-1,2,4-triazine;
3,5-Diamino-6-[3-(2,5 dichlorothienyl)]-1,2,4-triazine;
3,5-Diamino-6-[2-(3,4,5 trichlorothienyl)]-1,2,4-triazine;
5(3)-Amino-6-[3-(2,5-dichlorothienyl)]-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-ethyl-1,2,4-triazine;
3,5-Diamino-6-[2-(4,5-dibromothienyl)]-1,2,4-triazine;
3,5-Diamino-6-[2-(5-bromothienyl)]-1,2,4-triazine;
3,5-Diamino-6-[2-(3-bromothienyl)]-1,2,4-triazine;
3,5-Diamino-6-[2-(5-chlorothienyl)]-1,2,4-triazine;
3,5-Diamino-6-[2-(benzo[b]thienyl)]-1,2,4-triazine; and
3,5-Diamino-6-[2-(3-chlorobenzo[b]thienyl)]-1,2,4-triazine;
   or a salt, tautomer or solvate thereof;
   or the compound is selected from the group consisting of:
2,6-Diamino-3-(2-thienyl)-pyrazine;
2,4-Diamino-5-(2-thienyl)-pyrimidine;
2,6-Diamino-3-(3-thienyl)-pyrazine;
2,4-Diamino-5-(3-thienyl)-pyrimidine;
2,6-Diamino-3-[3-(2,5 dichlorothienyl)]-pyrazine;
2,4-Diamino-5-[3-(2,5 dichlorothienyl)]-pyrimidine;
2,6-Diamino-3-[2-(3,4,5 trichlorothienyl)]-pyrazine;
2,4-Diamino-5-[2-(3,4,5 trichlorothienyl)]-pyrimidine;
2(6)-Amino-3-(2-thienyl)-2,3(2,5)-dihydro-6(2)-imino-5-methyl-pyrazine;
4(2)-Amino-5-(2-thienyl)-2,3(2,5)-dihydro-2(4)-imino-1-methyl-pyrimidine;
2(6)-Amino-3-(2-thienyl)-2,3(2,5)-dihydro-6(2)-imino-5-ethyl-pyrazine;
4(2)-Amino-5-(2-thienyl)-2,3(2,5)-dihydro-2(4)-imino-1-ethyl-pyrimidine;
2(6)-Amino-3-[3-(2,5-dichlorothienyl)]-2,3(2,5)-dihydro-6(2)-imino-5-methyl-pyrazine;
4(2)-Amino-5-[3-(2,5-dichlorothienyl)]-2,3(2,5)-dihydro-2(4)-imino-2-methyl-pyrimidine;
2(6)-Amino-3-{2-(3,4,5-trichloro)thienyl}-2,3(2,5)-dihydro-6(2)-imino-5-methyl-pyrazine;
4(2)-Amino-5-{2-(3,4,5-trichloro)thienyl}-2,3(2,5)-dihydro-2(4)-imino-1-methyl-pyrimidine;
2(6)-Amino-3-{2-(3,4,5-trichloro)thienyl}-2,3(2,5)-dihydro-6(2)-imino-5-ethyl-pyrazine;
4(2)-Amino-5-{2-(3,4,5-trichloro)thienyl}-2,3(2,5)-dihydro-2(4)-imino-2-ethyl-pyrimidine;
2,6-Diamino-3-[2-(4,5-dibromothienyl)]-pyrazine;
2,4-Diamino-5-[2-(4,5-dibromothienyl)]-pyrimidine;
2,6-Diamino-3-[2-(5-bromothienyl)]-pyrazine;
2,4-Diamino-5-[2-(5-bromothienyl)]-pyrimidine;
2,6-Diamino-3-[2-(3-bromothienyl)]-pyrazine;
2,4-Diamino-5-[2-(3-bromothienyl)]-pyrimidine;
2,6-Diamino-3-[2-(5-chlorothienyl)]-pyrazine;
2,4-Diamino-5-[2-(5-chlorothienyl)]-pyrimidine;
2,6-Diamino-3-[2-(benzo[b]thienyl)]-pyrazine;
2,4-Diamino-5-[2-(benzo[b]thienyl)]-pyrimidine;
2,6-Diamino-3-[2-(3-chlorobenzo[b]thienyl)]-pyrazine; and
2,4-Diamino-5-[2-(3-chlorobenzo[b]thienyl)]-pyrimidine;
   or a salt, tautomer or solvate thereof.

As Embodiment 13, the invention provides a compound of formula (I), or a salt, tautomer or solvate thereof, for use as defined in any one of Embodiments 1 to 7, wherein A is a group of the formula

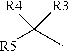

(wherein ⋅ indicates the point of attachment).
R3 is phenyl, xanthyl or naphthyl, each optionally substituted with 1 to 5 substituents selected from one or more halogen or C₁-C₆alkoxy groups;
R4 is selected from hydrogen, C₁-C₆alkyl, C₃-C₈cycloalkyl, phenyl, xanthyl or naphthyl, wherein the phenyl or naphthyl may be optionally substituted with 2 to 5 substituents selected from halogen or C₁-C₆alkoxy groups; and
R5 is hydrogen.

As Embodiment 14, the invention provides a compound of formula 1 or a salt, tautomer or solvate thereof, for use as defined in Embodiment 13, wherein R3 is phenyl, optionally substituted with 2 or 3 substituents selected from one or more halogen or C₁-C₆alkoxy groups; and

R4 is selected from C₁-C₆alkyl, C₃-C₈cycloalkyl, phenyl, wherein the phenyl may be optionally substituted with 2 to 3 substituents selected from halogen or C₁-C₆alkoxy groups.

As Embodiment 15, the invention provides a compound of formula 1 or a salt, tautomer or solvate thereof, for use as defined in Embodiment 14, wherein the compound is selected from the group consisting of:
3,5-Diamino-6-[1,1 bis-(4-chlorophenyl)methyl]-1,2,4-triazine;
3,5-Diamino-6-[1,1-bis-(4-fluorophenyl)methyl]-1,2,4-triazine;
3,5-Diamino-6-(diphenylmethyl)-1,2,4-triazine;
3,5-Diamino-6-(1-cyclopentyl-1-phenyl-methyl)-1,2,4-triazine;
3,5-Diamino-6-[1-(6-methoxynaphthalene)methyl]-1,2,4-triazine;
3,5-Diamino-6-[1-(6-methoxynaphthalene)ethyl]-1,2,4-triazine;
3,5-Diamino-6-(1-isopropyl-1-phenylmethyl)-1,2,4-triazine
3,5-Diamino-6-(9-xanthyl)-1,2,4-triazine; and
3,5-Diamino-6-{1-(4-chlorophenoxy)-1-methyl}ethyl-1,2,4-triazine;
   0r a salt, tautomer or solvate thereof;
   or the compound is selected from the group consisting of:
2,6-diamino-3-(diphenylmethyl)-pyrazine;
2,4-diamino-5-(diphenylmethyl)-pyrimidine;
2,6-Diamino-3-(1-cyclopentyl-1-phenyl-methyl)-pyrazine;
2,4-Diamino-5-(1-cyclopentyl-1-phenyl-methyl)-pyrimidine;
2,6-Diamino-3-[1-(6-methoxynaphthalene)methyl]-pyrazine;
2,4-Diamino-5-[1-(6-methoxynaphthalene)methyl]-pyrimidine;
2,6-Diamino-3-[1-(6-methoxynaphthalene)ethyl]-pyrazine;
2,4-Diamino-5-[1-(6-methoxynaphthalene)ethyl]-pyrimidine;
2,6-Diamino-3-(1-isopropyl-1-phenylmethyl)-pyrazine;
2,4-Diamino-5-(1-isopropyl-1-phenylmethyl)-pyrimidine;
2,6-Diamino-3-(9-xanthyl)-pyrazine;
2,4-Diamino-5-(9-xanthyl)-pyrimidine;
2,6-Diamino-3-[1,1 bis-(4-chlorophenyl)methyl]-pyrazine;
2,4-Diamino-5-[1,1 bis-(4-chlorophenyl)methyl]-pyrimidine;
2,6-Diamino-3-[1,1-bis-(4-fluorophenyl)methyl]-pyrazine;
2,4-Diamino-5-[1,1-bis-(4-fluorophenyl)methyl]-pyrimidine;
2,6-Diamino-3-{1-(4-chlorophenoxy)-1-methyl}ethyl-pyrazine; and
2,4-Diamino-5-{1-(4-chlorophenoxy)-1-methyl}ethyl-pyrimidine;
   or a salt, tautomer or solvate thereof.

As Embodiment 16, the invention provides a method of treating cytokine storm syndrome or cytokine release syndrome, comprising the step of administering to a subject in need thereof a therapeutically effective amount of a compound of formula (I) or a salt, tautomer or solvate thereof, wherein the compound of formula (I) is as defined in any preceding Embodiment.

As Embodiment 17, the invention provides the use of a compound of formula (I) or a salt, tautomer or solvate thereof, as defined hereinabove, in the manufacture of a medicament for use in the treatment of cytokine storm syndrome or cytokine release syndrome, wherein the compound of formula (I) is as defined in any one of Embodiments 1 to 15.

As Embodiment 18, invention further provides a pharmaceutical composition comprising a compound of formula (I) or a salt, tautomer or solvate thereof, and one or more pharmaceutically acceptable excipients for use in the treatment of cytokine storm syndrome or cytokine release syndrome, wherein the compound of formula (I) is as defined in any one of Embodiments 1 to 15.

The use of salts of the compounds of formula (I) form an aspect of this invention. Preferred salts are pharmaceutically acceptable acid addition salts. Suitable pharmaceutically acceptable acid addition salts include those formed with both organic and inorganic acids, for example from hydrochloric, sulphuric, citric, tartaric, phosphoric, lactic, pyruvic, acetic, malonic, succinic, oxalic, fumaric, maleic, oxaloacetic, methanesulphonic, p-toluenesulphonic, benzene-sulphonic, glutamic, naphthoic, and isethionic acids. Ethanesulfonate, malate, mandalate, benzoate, and salicylate salts are also suitable. Base addition salts also form an aspect of the invention.

In preparation of the compounds of formula (I), the compound or its salt may be obtained as a solvate of the reaction solvent or crystallisation solvent or a component thereof. Use of such solvates forms another aspect of this invention. Suitable pharmaceutically acceptable solvates include hydrates.

Certain compounds of structure (I) have chiral centres and may occur as racemates, racemic mixtures and as individual enantiomers or diastereomers. All such isomeric forms are included within the present invention. Also included within the scope of the invention are all geometric isomers of the compound of formula (I) whether as individual isomers or mixtures thereof. Thus, compounds of structure (I) in the trans and cis configuration form a further aspect of the invention; also all other tautomeric form of structure (I), including mixtures thereof. Furthermore, some of the crystalline forms of the compounds of structure (I) may exist as polymorphs, which are all included in the present invention.

Diazine compounds of Formula (I) may be prepared by procedures analogous to those described in EP-0372934A. The reactants of formulae (II), (IV) and (V) disclosed in EP-0372934A may be replaced with corresponding sulphur containing heterocyclic analogues in order to prepare compounds useful in the present invention.

Alternatively, compounds of Formula (I) may be prepared according to the procedures described in WO2009090431A.

The preparation of specific compounds mentioned above is illustrated later in this specification. Related compounds within the scope of the invention may be prepared by obvious or routine variations of the disclosed processes, using appropriate starting materials to introduce the desired substituents and moieties of compounds within the scope of formula (I).

Salts of compounds of formula (I) may be obtained by the presence of a residual acid in the preparative process. Alternatively, salts may be prepared by mixing the compound of formula (I) as the free base with a pharmaceutically acceptable acid in a suitable solvent, and removing the solvent to recover the salt, or crystallising the salt from the solvent.

In a further aspect, the present invention provides pharmaceutical compositions for the treatment of disorders such as cytokine storm syndrome or cytokine release syndromes, or a pharmaceutically acceptable salt, tautomer or solvate thereof, in admixture with one or more pharmaceutically acceptable excipients.

The compounds of formula (I) will be present in the compositions of the present invention in an effective unit dosage form, that is to say in an amount sufficient to be effective against the disorders in vivo.

The pharmaceutically acceptable carriers present in the compositions of the present invention may be materials conventionally used for the purpose of administering the medicament. These may be liquid or solid materials, which are otherwise inert or medically acceptable and are compatible with the active ingredients.

These pharmaceutical compositions may be given orally or parenterally, for example as a suppository, ointment, cream, powder or trans-dermal patch. However, oral administration and intravenous injection of the compositions are preferred.

For oral administration, fine powders or granules will contain diluting, dispersing and/or surface active agents, and may be presented in draught, in water or in a syrup, in capsules or sachets in the dry state or in non-aqueous suspension wherein suspending agents may be included, or in a suspension in water or syrup. Where desirable or necessary, flavouring, preserving, suspending, or thickening agents can be included. Dry powders or granules may be compressed to form a tablet or contained in a capsule.

For injection, the compounds may be presented in sterile aqueous injection solutions which may contain anti-oxidants or buffers.

The free base or a salt or solvate thereof may also be administered in its pure form unassociated with other additives in which case a capsule or sachet is the preferred carrier.

Alternatively, the active compound may be presented in a pure form as an effective unit dosage, for instance compressed as a tablet or the like.

Other compounds which may be included are, for example, medically inert ingredients, e.g., solid and liquid diluents such as lactose, starch, or calcium phosphate for tablet or capsules; olive oil or ethyl oleate for soft capsules; and water or vegetable oil for suspensions or emulsions; lubricating agents such as talc or magnesium stearate; gelling agents such as colloidal clays; thickening agents such as gum tragacanth or sodium alginate; and other therapeutically acceptable accessory ingredients such as humectants, preservatives, buffers, and antioxidants which are useful as carriers in such formulations.

Tablets or other forms of presentation provided in discrete units may conveniently contain an amount of compound of formula I which is effective at such dosage or as a multiple of the same, for instance units containing 5 mg to 500 mg, usually around 10 mg to 250 mg.

The pharmaceutical compositions of the present invention may be prepared by the admixture of a compound of formula (I) with a pharmaceutically acceptable carrier. Conventional pharmaceutical excipients may be admixed as required. Example of suitable formulations are discussed in U.S. Pat. No. 4,649,139.

As indicated above, the compounds of formula (I) are generally useful in treating such disorders by oral administration or intravenous injection.

The compounds of formula (I) are normally administered at a dose of from 0.01 mg/kg to 20 mg/kg per day, preferably 0.1 to 5.0 mg/kg per day.

WO2016/198878A1 discloses Interferon-gamma (IFN-γ) inhibitory activity for certain compounds useful in the present invention. It has now been found that the diazine and triazine compounds of the present invention also possess activity as inhibitors of TNF-α, and Interleukin-1β, 2, 4, 6, 8, 13 and 17, as detailed below. Due to this combination of therapeutic activity therefore, the compounds are useful for the treatment of cytokine storm syndrome and cytokine release syndrome.

### EXPERIMENTAL

The compounds of Formula (I) may be prepared according to the methods disclosed in WO2009/090431A1, using the appropriate starting materials.

### Example 1: 2,4-Diamino-5-(diphenylmethyl)-pyrimidine

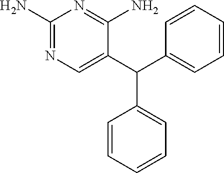 [SMILES: Nc1ncc(C(c2ccccc2)c2ccccc2)c(N)n1]

Formula: C17H16N4

Molecular weight: 276.34

LCMS: m/z=277.20, consistent for protonated parent ion (M+H)⁺

¹H NMR (DMSO-d6): The ¹H NMR spectrum was found to be consistent with the above structure.

Purity: >99% by HPLC

The compounds of Formula (I) may be investigated for inhibition of pro-inflammatory cytokines Interleukin (IL)-10, 6,8 and 17A, Interferon (IFN)-gamma and Tumour necrosis factor (TNF)-α in peripheral blood mononuclear cells (PBMCs) isolated from fresh human buffy coats by centrifugation on Lymphoprep™ (Stemcell Technologies). All human cells are grown in cell culture medium RPMI-1640 supplemented with 1% penicillin/streptomycin and 5% heat inactivated fetal bovine serum.

PBMCs stimulated with LPS (Salmonella enterica serotype typhimurium) are incubated for 24 hours containing the compounds under investigation, reconstituted in dimethyl sulfoxide (DMSO). The secreted levels of Interleukin-1β and Interleukin-6 are measured in the cell culture supernatant using a cytometric bead array and the cell viability is quantified using Trypan blue.

PBMCs stimulated with a mixture between TNF-α and IL-17A are incubated for 24 hours whilst containing the compounds under investigation, reconstituted in DMSO. The secreted levels of Interleukin-8 are measured in the cell culture supernatant using a cytometric bead array and the cell viability is quantified using Trypan blue.

PBMCs stimulated with PMA/ionomycin are incubated for 24 hours containing the compounds under investigation, reconstituted in DMSO. The secreted levels of Interleukin-17A are measured in the cell culture supernatant using a cytometric bead array and the cell viability is quantified using Trypan blue.

PBMCs stimulated with PMA/ionomycin are incubated for 24 hours containing the compounds under investigation, reconstituted in DMSO. The secreted levels of Interferon-gamma and Tumour necrosis factor-α are measured in the cell culture supernatant using a Human Quantikine ELISA Kit and the cell viability is quantified using Trypan blue.

The compounds may be investigated for inhibition of pro-inflammatory cytokines Interleukin-2, 4 and 13 in human CD4-positive T cells isolated from fresh isolated PBMCS using a CD4-positive T cell isolation kit.

CD4-positive T cells stimulated with beads coated with antibodies against CD2, CD3 and CD28 are incubated for 48 hours containing the compounds under investigation, reconstituted in DMSO. The secreted levels of Interleukin-2, 4 and 13 are measured in the cell culture supernatant using a cytometric bead array and the cell viability is measured using an MTT (3-(4,5-dimethylthiazol-2-yl)-2,5-diphenyltetrazolium bromide) assay.

Secreted IL-4 levels may be measured using electrochemiluminescence (MSD kits, Meso Scale Discovery), while secreted IL-2 levels may be measured using proximity homogenous time-resolved fluorescence (HTRF) and the amount of living cells may be measured by addition of resazurin (PrestoBlue®).

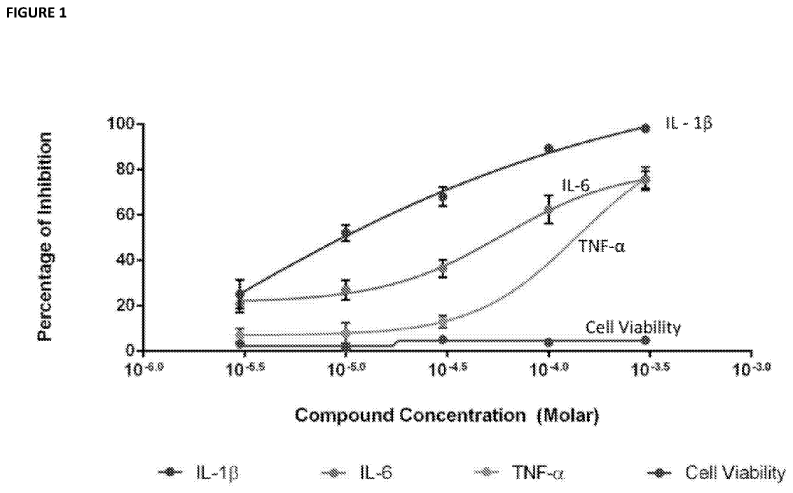

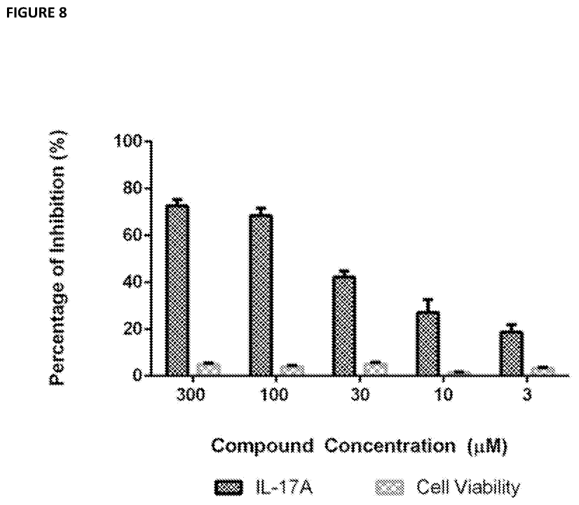

Compounds 1, 3,5-diamino-6-[2-(3,4,5-trichlorothienyl)]-1,2,4-triazine, and Compound 2, 3,5-Diamino-6-(diphenylmethyl)-1,2,4-triazine, were investigated using the methods described above. Compound 1 was found to exhibit high inhibition of the proinflammatory cytokines IL-1β, IL-8, IL-6, IFN-γ, TNF-α as well as moderate inhibition of IL-17, as shown in FIGS. 1 to 7. Compound 2 was found to exhibit high inhibition of IL-17A, IL-8, IFN-γ, TNF-α (at the top concentration) and a moderate inhibition of IL-1β and IL-6, as shown in FIGS. 8 to 12.

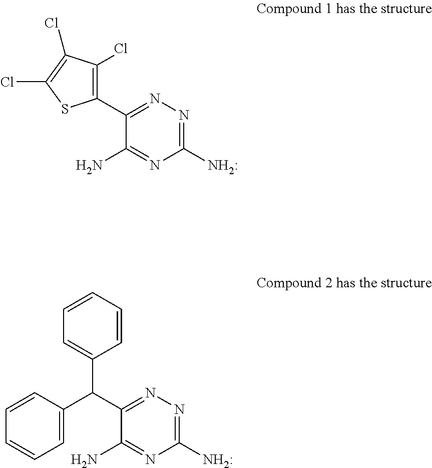

Compounds 1 and 2 may be prepared by the processes disclosed in WO2009/090431A1. When tested in IL-2 and IL-4 inhibition assays, 3,5-Diamino-6-(diphenylmethyl)-1,2,4-triazine was found to exhibit relative EC50 of 28.0 and 50.5 nM respectively:

| | Rel EC50 | | Abs EC50 | | Max Effect |
|---|---|---|---|---|---|
| Inhibition of IL-2 release | 28.0 | nM | 15.0 | nM | 97% |
| Inhibition of IL-4 release | 58.4 | nM | 61.3 | nM | 99% |
| Inhibition of T cell | >10000.0 | nM | >10000.0 | nM | 28% |
| proliferation | | | | | |

These data evidences a good level of inhibition of both IL-2 and IL-4 by 3,5-Diamino-6-(diphenylmethyl)-1,2,4-triazine without significant inhibition of T cell proliferation.

3,5-Diamino-6-(diphenylmethyl)-1,2,4-triazine was also investigated for IL-7A inhibition in a human PBMC model:

| | | |
|---|---|---|
| Rel EC50 | Abs EC50 | Max effect |

| | | | | | |
|---|---|---|---|---|---|
| Inhibition of IL-17A | 261 | nM | 317 | nM | 86% |
| release | | | | | |
| Inhibition of PMBC | 44000 | nM | >10000 | nM | 38% |
| viability | | | | | |

Further data are shown in the Figures:

FIG. 1 illustrates the effects of compound 1 on cytokine production from stimulated human peripheral blood mononuclear cells, wherein the bars indicate a mean±SEM for n=9-12 subjects.

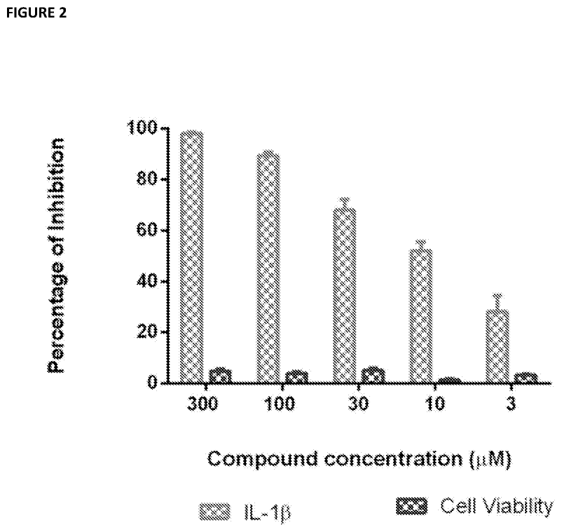

FIG. 2 illustrates the inhibitory effect of compound 1 on Interleukin (IL)-1 beta, wherein the bars indicate a mean±SEM for n=9-10 subjects.

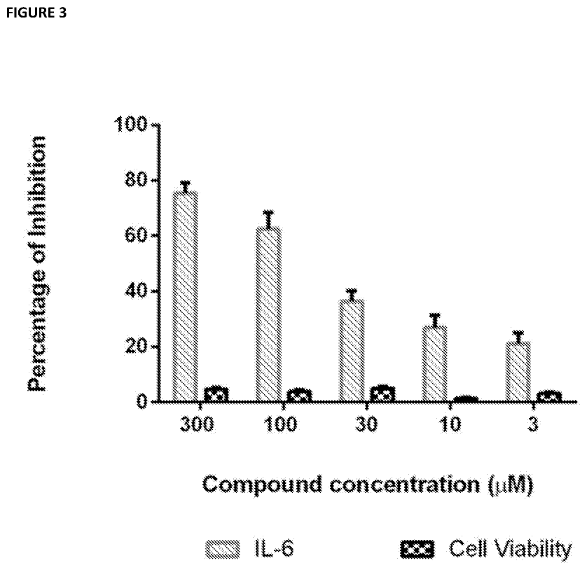

FIG. 3 illustrates the inhibitory effect of compound 1 on interleukin (IL)-6, wherein the bars indicate a mean±SEM for n=9-10 subjects.

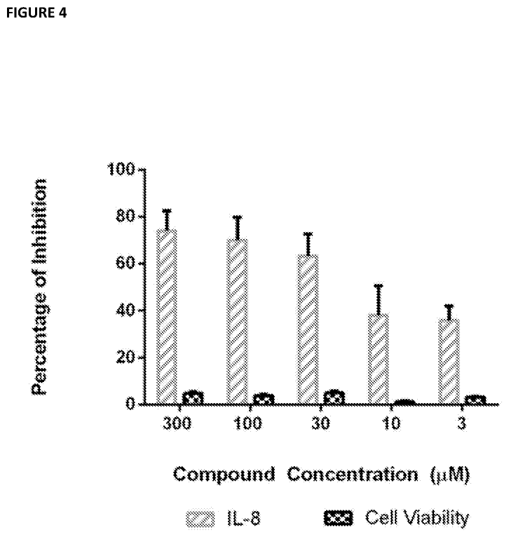

FIG. 4 illustrates the inhibitory effect of compound 1 on interleukin (IL)-8, wherein the bars indicate a mean±SEM for n=8-12 subjects.

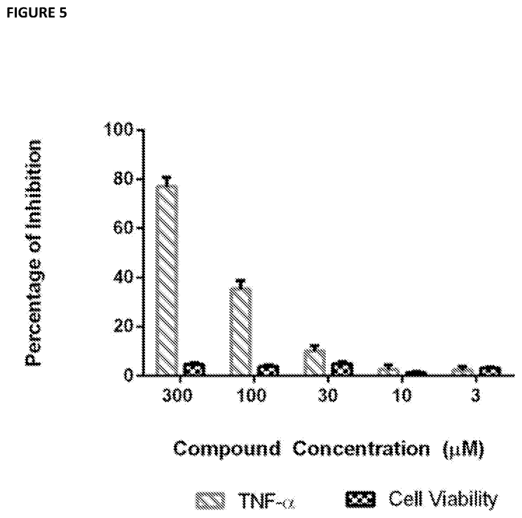

FIG. 5 illustrates the inhibitory effect of compounds 1 on Tumour Necrosis Factor (TNF)-α, wherein the bars indicate a mean±SEM for n=8-12 subjects.

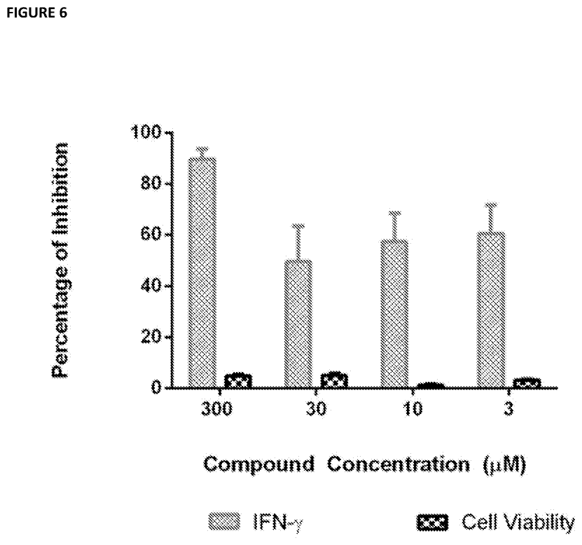

FIG. 6 illustrates the inhibitory effect of compound 1 on interferon-gamma (IFN-γ), wherein the bars indicate a mean±SEM for n=5-7 subjects.

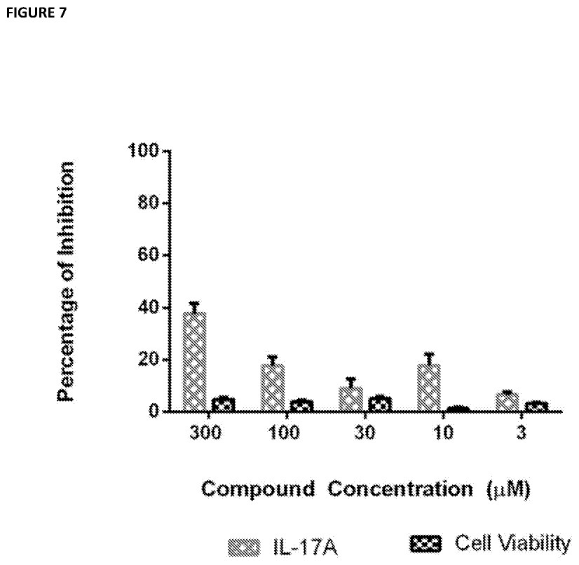

FIG. 7 illustrates the inhibitory effect of compound 1 on interleukin (IL)-17A, wherein the bars indicate a mean±SEM for n=5-7 subjects.

FIG. 8 illustrates the inhibitory effect of compound 2 on interleukin (IL)-17A, wherein the bars indicate a mean±SEM for n=9-12 subjects.

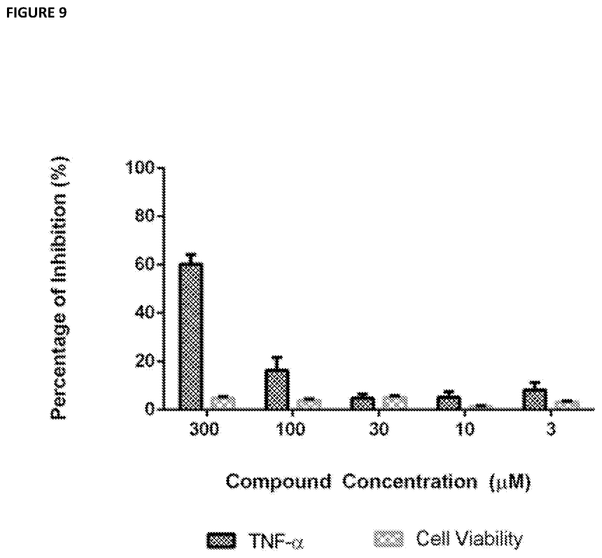

FIG. 9 illustrates the inhibitory effect of compound 2 on Tumour Necrosis Factor (TNF)-α, wherein the bars indicate a mean±SEM for n=9-12 subjects.

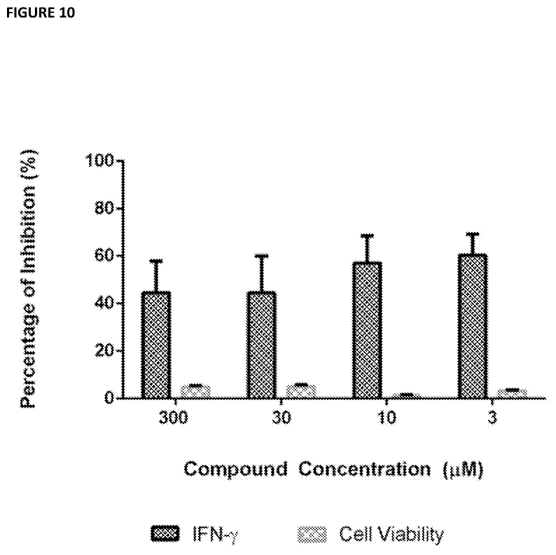

FIG. 10 illustrates the inhibitory effect of compound 2 on interferon-gamma (IFN-γ), wherein the bars indicate a mean±SEM for n=5-8 subjects.

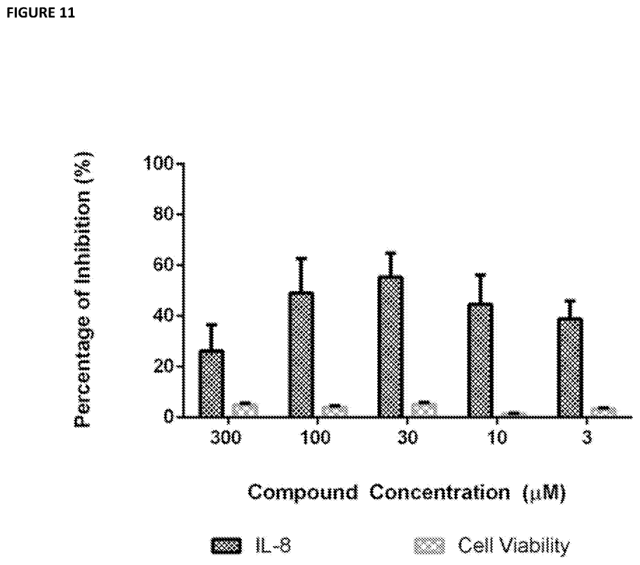

FIG. 11 illustrates the inhibitory effect of compound 2 on interleukin (IL)-8, wherein the bars indicate a mean±SEM for n=5-8 subjects.

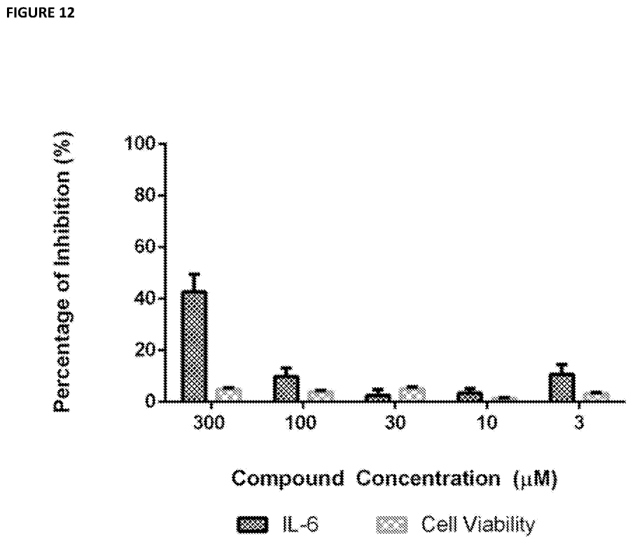

FIG. 12 illustrates the inhibitory effect of compound 2 on interleukin (IL)-6, wherein the bars indicate a mean±SEM for n=9-10 subjects.

1. A compound of Formula (I), or a salt, tautomer or solvate thereof, 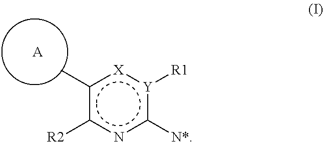
in which:
X is N and Y is C; or
X is C and Y is N; or
X and Y are both N:
A is a substituted 3 to 10 membered heterocycle comprising one, two or three sulphur atoms; said heterocycle having two or more substituents selected from (i) halogen; (ii) C₁₋₆ alkyl, C₂₋₆ alkenyl, C₂₋₆ alkynyl, or C₁₋₆ alkoxy, all optionally substituted by one or more of halogen, hydroxy and aryl; and (iii) amino, mono- or di-substituted amino, alkenyloxy, acyl, acyloxy, cyano, nitro, aryl and C₁₋₆alkylthio groups; or
A is a group 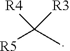
(wherein ⋅ indicates the point of attachment)
R1 is hydrogen, or a substituent group selected from C₁₋₁₀ alkyl, C₂₋₁₀ alkenyl, benzyl, piperidine-methyl, thienyl-methyl, furyl-methyl or C₃₋₁₀cycloalkyl, any of which is optionally substituted by hydroxy, halogen, carboxamide, halo C₁₋₆alkyl, C₁₋₆ alkyl or C₁₋₆ alkoxy; or the Y is N and is unsubstituted;
R2 is amino, C₁₋₁₀ alkyl or phenyl;
R3 is phenyl, xanthyl or naphthyl, each optionally substituted with 1 to 5 substituents selected from halogen or C₁-C₆ alkoxy groups,
R4 is selected from hydrogen, C₁-C₆ alkyl, C₃-C₈ cycloalkyl, phenyl, xanthyl or naphthyl, wherein the phenyl or naphthyl may be optionally substituted with 2 to 5 substituents selected from halogen or C₁-C₆ alkoxy groups;
R5 is hydrogen;
and
N* is ═NH when R1 is hydrogen or a substituent group; or
N* is a group NRaRb where Ra and Rb are independently H or an alkyl group; or
N* is a piperazinyl ring, optionally substituted with one or more halogen or C₁-C₆alkoxy groups;
for use in the treatment of cytokine storm syndrome or cytokine release syndrome.

2. A compound of Formula (I), or a salt, tautomer or solvate thereof, as defined in claim 1, for use in the treatment of cytokine storm syndrome triggered by an infection.

3. A compound of Formula (I), or a salt, tautomer or solvate thereof, as defined in claim 1, for use in the treatment of cytokine storm syndrome triggered by an infection selected from sepsis, influenza virus, coronavirus, and dengue virus.

4. A compound of Formula (I), or a salt, tautomer or solvate thereof, as defined in claim 1, for use in the treatment of cytokine storm syndrome triggered by a coronavirus.

5. A compound of Formula (I), or a salt, tautomer or solvate thereof, as defined in claim 1, wherein X and Y are both N.

6. A compound of Formula (I), or a salt, tautomer or solvate thereof, as defined in claim 1, wherein R1 is hydrogen.

7. A compound of Formula (I), or a salt, tautomer or solvate thereof, as defined in claim 1, wherein R2 is amino.

8. A compound of Formula (I), or a salt, tautomer or solvate thereof, as defined in claim 1, wherein A is a substituted 3 to 10 membered heterocycle comprising one, two or three sulphur atoms; said heterocycle having two or more substituents selected from (i) halogen; (ii) C₁₋₆ alkyl, C₂₋₆ alkenyl, C₂₋₆ alkynyl, or C₁₋₆alkoxy, all optionally substituted by one or more of halogen, hydroxy and aryl; and (iii) amino, mono- or di-substituted amino, alkenyloxy, acyl, acyloxy, cyano, nitro, aryl and C₁₋₆ alkylthio groups.

9. A compound of formula (I), or a salt, tautomer or solvate thereof, as defined in claim 1, wherein A is thienyl, or benzothienyl.

10. A compound of formula (I), or a salt, tautomer or solvate thereof, as defined in claim 8, wherein A is substituted with one or more substituents selected from halogen, C₁₋₆ alkyl, C₁₋₆ alkoxy, haloC₁₋₆ alkyl and haloC₁₋₆alkoxy.

11. A compound of formula (I), or a salt, tautomer or solvate thereof, as defined claim 1, wherein A is substituted with 1, 2, or 3 chlorine or bromine atoms.

12. A compound of formula (I), or a salt, tautomer or solvate thereof, as defined in claim 1, wherein the compound is selected from the group consisting of:
3.5-Diamino-6-(2-thienyl)-1,2,4-triazine;
3.5-Diamino-6-(3-thienyl)-1,2,4-triazine;
5(3)-Amino-6-(2-thienyl)-2,3(2,5)-dihydro-3(5)-imino-2-methyl-1,2,4-triazine methanesulfonate;
5(3)-Amino-6-(2-thienyl)-2,3(2,5)-dihydro-3(5)-imino-2-ethyl-1,2,4-triazine methanesulfonate;
3.5-Diamino-6-(2-thienyl)-1,2,4-triazine:
3.5-Diamino-6-(3-thienyl)-1,2,4-triazine;
3.5-Diamino-6-[3-(2,5 dichlorothienyl)]-1,2,4-triazine;
3.5-Diamino-6-[2-(3,4,5 trichlorothienyl)]-1,2,4-triazine;
5(3)-Amino-6-[3-(2,5-dichlorothienyl)]-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-methyl-1,2,4-triazine;
5(3)-Amino-6-{2-(3,4,5-trichloro)thienyl}-2,3(2,5)-dihydro-3(5)-imino-2-ethyl-1,2,4-triazine;
3.5-Diamino-6-[2-(4,5-dibromothienyl)]-1,2,4-triazine:
3.5-Diamino-6-[2-(5-bromothienyl)]-1,2,4-triazine;
3.5-Diamino-6-[2-(3-bromothienyl)]-1,2,4-triazine;
3.5-Diamino-6-[2-(5-chlorothienyl)]-1,2,4-triazine;
3.5-Diamino-6-[2-(benzo[b]thienyl)]-1,2,4-triazine; and
3.5-Diamino-6-[2-(3-chlorobenzo[b]thienyl)]-1,2,4-triazine;
or a salt, tautomer or solvate thereof;
or the compound is selected from the group consisting of:
2.6-Diamino-3-(2-thienyl)-pyrazine;
2.4-Diamino-5-(2-thienyl)-pyrimidine;
2.6-Diamino-3-(3-thienyl)-pyrazine;
2.4-Diamino-5-(3-thienyl)-pyrimidine;
2.6-Diamino-3-[3-(2,5 dichlorothienyl)]-pyrazine;
2.4-Diamino-5-[3-(2,5 dichlorothienyl)]-pyrimidine;
2.6-Diamino-3-[2-(3,4,5 trichlorothienyl)]-pyrazine;
2.4-Diamino-5-[2-(3,4,5 trichlorothienyl)]-pyrimidine;
2(6)-Amino-3-(2-thienyl)-2,3(2,5)-dihydro-6(2)-imino-5-methyl-pyrazine;
4(2)-Amino-5-(2-thienyl)-2,3(2,5)-dihydro-2(4)-imino-1-methyl-pyrimidine;
2(6)-Amino-3-(2-thienyl)-2,3(2,5)-dihydro-6(2)-imino-5-ethyl-pyrazine;
4(2)-Amino-5-(2-thienyl)-2,3(2,5)-dihydro-2(4)-imino-1-ethyl-pyrimidine;
2(6)-Amino-3-[3-(2,5-dichlorothienyl)]-2,3(2,5)-dihydro-6(2)-imino-5-methyl-pyrazine;
4(2)-Amino-5-[3-(2,5-dichlorothienyl)]-2,3(2,5)-dihydro-2(4)-imino-2-methy-pyrimidine;
2(6)-Amino-3-{2-(3,4,5-trichloro)thienyl}-2,3(2,5)-dihydro-6(2)-imino-5-methyl-pyrazine;
4(2)-Amino-5-{2-(3,4,5-trichloro)thienyl}-2,3(2,5)-dihydro-2(4)-imino-1-methyl-pyrimidine;
2(6)-Amino-3-{2-(3,4,5-trichloro)thienyl}-2,3(2,5)-dihydro-6(2)-imino-5-ethyl-pyrazine;
4(2)-Amino-5-{2-(3,4,5-trichloro)thienyl}-2,3(2,5)-dihydro-2(4)-imino-2-ethyl-pyrimidine;
2.6-Diamino-3-[2-(4,5-dibromothienyl)]-pyrazine;
2.4-Diamino-5-[2-(4,5-dibromothienyl)]-pyrimidine;
2.6-Diamino-3-[2-(5-bromothienyl)]-pyrazine;
2.4-Diamino-5-[2-(5-bromothienyl)]-pyrimidine;
2.6-Diamino-3-[2-(3-bromothienyl)]-pyrazine:
2.4-Diamino-5-[2-(3-bromothienyl)]-pyrimidine;
2.6-Diamino-3-[2-(5-chlorothienyl)]-pyrazine;
2.4-Diamino-5-[2-(5-chlorothienyl)]-pyrimidine;
2.6-Diamino-3-[2-(benzo[b]thienyl)]-pyrazine;
2.4-Diamino-5-[2-(benzo[b]thienyl)]-pyrimidine;
2.6-Diamino-3-[2-(3-chlorobenzo[b]thienyl)]-pyrazine; and
2.4-Diamino-5-[2-(3-chlorobenzo[b]thienyl)]-pyrimidine;
or a salt, tautomer or solvate thereof.

13. A compound of formula (I), or a salt, tautomer or solvate thereof, as defined in claim 1, wherein A is a group of the formula 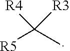
(wherein ⋅ indicates the point of attachment)
R3 is phenyl, xanthyl or naphthyl, each optionally substituted with 1 to 5 substituents selected from one or more halogen or C₁-C₆alkoxy groups;
R4 is selected from hydrogen, C₁-C₆alkyl, C₃-C₈cycloalkyl, phenyl, xanthyl or naphthyl, wherein the phenyl or naphthyl may be optionally substituted with 2 to 5 substituents selected from halogen or C₁-C₆alkoxy groups, and
R5 is hydrogen.

14. A compound of formula 1 or a salt, tautomer or solvate thereof, as defined in claim 13, wherein R3 is phenyl, optionally substituted with 2 or 3 substituents selected from one or more halogen or C₁-C₆alkoxy groups; and
R4 is selected from C₁-C₆alkyl, C₃-C₈cycloalkyl, phenyl, wherein the phenyl may be optionally substituted with 2 to 3 substituents selected from halogen or C₁-C₆alkoxy groups.

15. A compound of formula 1 or a salt, tautomer or solvate thereof, as defined in claim 14, wherein the compound is selected from:
3.5-Diamino-6-[1,1 bis-(4-chlorophenyl)methyl]-1,2,4-triazine;
3.5-Diamino-6-[1,1-bis-(4-fluorophenyl)methyl]-1,2,4-triazine:
3.5-Diamino-6-(diphenylmethyl)-1,2,4-triazine;
3.5-Diamino-6-(1-cyclopentyl-1-phenyl-methyl)-1,2,4-triazine;
3.5-Diamino-6-[1-(6-methoxynaphthalene)methyl]-1,2,4-triazine;
3.5-Diamino-6-[1-(6-methoxynaphthalene)ethyl]-1,2,4-triazine;
3.5-Diamino-6-(1-isopropyl-1-phenylmethyl)-1,2,4-triazine
3.5-Diamino-6-(9-xanthyl)-1,2,4-triazine;
3.5-Diamino-6-{1-(4-chlorophenoxy)-1-methyl}ethyl-1,2,4-triazine; and
2.4-Diamino-5-(diphenylmethyl)-pyrimidine; or
a salt, tautomer or solvate thereof;
or the compound is selected from the group consisting of:
2.6-diamino-3-(diphenylmethyl)-pyrazine;
2.4-diamino-5-(diphenylmethyl)-pyrimidine;
2.6-Diamino-3-(1-cyclopentyl-1-phenyl-methyl)-pyrazine;
2.4-Diamino-5-(1-cyclopentyl-1-phenyl-methyl)-pyrimidine;
2.6-Diamino-3-[1-(6-methoxynaphthalene)methyl]-pyrazine;
2.4-Diamino-5-[1-(6-methoxynaphthalene)methyl]-pyrimidine:
2.6-Diamino-3-[1-(6-methoxynaphthalene)ethyl]-pyrazine;
2.4-Diamino-5-[1-(6-methoxynaphthalene)ethyl]-pyrimidine;
2.6-Diamino-3-(1-isopropyl-1-phenylmethyl)-pyrazine;
2.4-Diamino-5-(1-isopropyl-1-phenylmethyl)-pyrimidine;
2.6-Diamino-3-(9-xanthyl)-pyrazine;
2.4-Diamino-5-(9-xanthyl)-pyrimidine;
2.6-Diamino-3-[1,1 bis-(4-chlorophenyl)methyl]-pyrazine;
2.4-Diamino-5-[1,1 bis-(4-chlorophenyl)methyl]-pyrimidine;
2.6-Diamino-3-[1,1-bis-(4-fluorophenyl)methyl]-pyrazine;
2.4-Diamino-5-[1,1-bis-(4-fluorophenyl)methyl]-pyrimidine;
2.6-Diamino-3-{1-(4-chlorophenoxy)-1-methyl}ethyl-pyrazine; and
2.4-Diamino-5-{1-(4-chlorophenoxy)-1-methyl}ethyl-pyrimidine;
or a salt, tautomer or solvate thereof.

16. A method of treating cytokine storm syndrome or cytokine release syndrome, comprising the step of administering to a subject in need thereof a therapeutically effective amount of a compound of formula (I) or a salt, tautomer or solvate thereof, wherein the compound of formula (I) is as defined in claim 1.

17. A pharmaceutical composition comprising a compound of formula (I) or a salt, tautomer or solvate thereof, and one or more pharmaceutically acceptable excipients for use in the treatment of cytokine storm syndrome or cytokine release syndrome, wherein the compound of formula (I) is as defined in claim 1.
